(19) United States
(12) Patent Application Publication
(10) Pub. No.: US 2025/0000928 A1
(43) Pub. Date: Jan. 2, 2025

(54) PERSIMMON DIETARY FIBER FOR ITS USE WITH OTHER BENEFICIAL MICROORGANISMS

(71) Applicant: UNIVERSIDAD MIGUEL HERNANDEZ DE ELCHE, Elche (ES)

(72) Inventors: Domingo SAURA LOPEZ, Elche (ES); Nuria MARTI BRUÑA, Elche (ES); Manuel VALERO ROCHE, Elche (ES); Maria Concepcion MARTINEZ MADRID, Elche (ES); Bryan MORENO CHAMBA, Elche (ES); Juuo SALAZAR BERMEO, Elche (ES)

(21) Appl. No.: 18/069,452

(22) Filed: Dec. 21, 2022

(30) Foreign Application Priority Data
Dec. 21, 2021 (ES) P202131183

(51) Int. Cl. A61K 36/44; A61K 35/744; A61K 35/745; A61K 35/747; A61P 31/04

(52) U.S. Cl. CPC A61K 36/44; A61K 35/744; A61K 35/745; A61K 35/747; A61P 31/04; A61K 2236/33; A61K 2236/37; A61K 2236/53

(57) ABSTRACT

Dietary fiber derived from persimmon by-products characterized in that it is obtained by means of a purification or extraction process assisted with acetone as solvent.

### REFERENCE TO RELATED PATENT APPLICATIONS

This application claims priority from patent application no P20213118312-21 filed with the Spanish patent office on Dec. 21, 2021 and which is incorporated herein in its entirety.

### DESCRIPTION

Obtaining and purification of dietary fiber from persimmon industrialization by-products and its selective combination with probiotic bacteria for pathogen displacement.

### FIELD OF THE ART

The present invention relates to a dietary fiber of persimmon industrial by-product which, after a purification process, can be combined with microorganisms considered beneficial with pathogen-displacing effects.

### STATE OF THE ART

Persimmon (Diospyros kaki Thunb.), a fleshy fruit belonging to the family Ebenaceae and native to China, is widely spread in countries such as Korea, Japan, Brazil. Italy and Spain (Lucas-González et al., 2018; Rodríguez-Garayar et al., 2017). In the latter, its initial cultivation was ornamental until it developed into a food consumption crop with almost 500 thousand tons of production until 2019 (FAOSTAT, 2021).

Persimmon presents nutritional importance due to its high content of bioactive compounds such as polyphenols, carotenoids, and dietary polysaccharides (Song et al., 2019). These compounds have been linked to the reduction of human degenerative diseases due to their antioxidant properties. Among these, cardiovascular diseases and metabolism-related disorders have been able to be combated with these compounds with results such as anti-diabetic properties, cholesterol lowering, serum glucose reduction, among others (López-Vargas et al., 2013; Akter et al., 2010; López-Marcos et al., 2015).

Dietary fiber plays a fundamental role in the human diet, that is, after being ingested. The benefits after its intake range from decreasing intestinal transit time, increasing fecal volume, softening stool, delaying gastric emptying, slowing glucose absorption, improving immune function, regulating the human microbiome, inhibiting hepatic cholesterol synthesis, among others (Sungsoo Cho & Dreher, 2001; Benito-González et al., 2019).

Other effects related to dietary fiber intake are its effect against autoimmune diseases, intestinal diseases and obesity, stimulating intestinal barrier function and inflammatory response (Chen et al., 2017; Hung & Suzuki, 2018; Lépine & De Vos, 2018). By interacting with the microbiome directly, fiber exerts a prebiotic effect after fermentation, promoting the production of short-chain fatty acids, release of polysaccharide matrix-bound compounds, antioxidant effects, stimulation of cell differentiation and apoptosis, color motility and optimization of absorption of electrolytes and other compounds, as well as regulation of the intestinal microbial population (Yamashita et al., 2003; Ueda et al., 2013; Kamimoto et al., 2014; Kim et al., 2017; Zhu, 2018; Benito-Gonzalez et al., 2019; Cruz-Requena et al., 2019).

The consumption of dietary fiber in clinical studies has shown positive effects. The consumption of 15 g/day of dietary fiber for 12 weeks among adults (29 to 59 years) with a BMI of 32.0±5.9 kg/m2, originated a significant loss of 0.87±0.37 kg of body mass, a decrease in appetite (16%) and in their serum glucose levels, as well as an increase in insulin levels compared to the placebo group (Lambert et al., 2017). Another study observed that fiber consumption for 12 weeks generated a significant alteration of short-chain fatty acids and bile acids (increased acetate and reduced isovalerate, cholate, deoxycholate and total bile acids). The microbiome was modulated following fiber consumption compared to the placebo group, with significant action of fiber on communities of potentially beneficial microorganisms observed (Mayengham et al., 2019).

Although the benefits of fiber consumption have been mentioned, it has been documented that the usual diet does not encompass the recommended amount of daily intake (25-35 g/day) (FAO, 2011). Several efforts have been made to incorporate more fiber in the diet and its combination with beneficial microorganisms has generated the concept of symbiotic. This combination has been shown to generate anti-inflammatory and pathogen displacement effects among others (Kazemi et al., 2020).

Our invention focuses on obtaining dietary fiber that, after a purification process, manifests a functionality that contributes to human health after ingestion. By undergoing a purification process, a change in its composition, microstructure, physicochemical, physicochemical, physio-functional, and physiological properties is generated (Akter et al., 2010; Martínez-Las Heras et al., 2017; Xiao et al., 2017; Bass et al., 2020), both in humans and in their microbiome.

The obtaining of dietary fiber from vegetables and by-products is carried out by different methods, such as the physical breaking of the vegetable cell wall, enzymatic treatments, fractionation with acids, application of ultrasound waves, drying treatments, among others (Patents US20180279655A1, CN106473159A, CN106414506A, CN101797038B, JP2013035803A). The application of solvents allows the removal of easily extractable compounds, increasing the surface reactivity of the treated polysaccharides and the scalability of the process.

As far as the persimmon by-product is concerned, there is no record of the utilization of its polysaccharides after purification treatment, especially solvents. Even so, the utilization of its by-product has been the subject of different patents of interest (Patents CN101327245-A, CN101468084-A, JP7145595-A, KR101314167B1, JP2013035803A, CN107594418A). Likewise, no combinations of these polysaccharides with other microorganisms have been reported.

The process object of the present invention is based on the application of a procedure with organic solvents for the extraction and purification of these compounds from by-products of the persimmon industry. The process subjects the by-product of the fruit to a solid phase process at a given rate, temperature, and time, eliminating easily extractable compounds from the by-product. The solvents used in this process are commonly used in the food industry. Probiotics that demonstrate an affinity for purified persimmon polysaccharides are commonly used in probiotic supplements and are commonly found in the human microbiome.

### REFERENCES

Akter, S., Ahmed, M., & Eun, J. B. (2010). Effect of blanching and drying temperatures on the physicochemical characteristics, dietary fiber composition and antioxidant-related parameters of dried persimmons peel powder. International Journal of Food Sciences and Nutrition, 61 (7), 702-712.
Beermann, C., Gruschwitz, N., Walkowski, K., & Göpel, A. (2013). Growth modulating properties of polyphenolic apple pomace extract on food associated microorganisms. Journal of Microbiology, Biotechnology and Food Services, 3 (2), 176-181.
Benito-González, I., Martínez-Sanz, M., Fabra, M. J., & López-Rubio, A. (2019). Health effect of dietary fibers. En Dietary Fiber: Properties, Recovery and Applications (pp. 125-163). Academic Press.
Carlson, J. L., Erickson, J. M., Hess, J. M., Gould, T. J., & Slavin, J. L. (2017). Prebiotic dietary fiber and gut health: comparing the in vitro fermentations of beta-glucan, inulin and xylooligosaccharide. Nutrients, 9 (12), 1361.
Chen, T., Kim, C. Y., Kaur, A., Lamothe, L., Shaikh, M., Keshavarzian, A., & Hamaker, B. R. (2017). Dietary fibre-based SCFA mitures promote both protection and repair of intestinal epithelial barrier function in a Caco-2 cell model. Food & Function, 8 (3), 1166-1173.
Cruz-Requena, M., Escobedo-García, S., Salas-Tovar, J. A., Mora-Cura, Y., Chávez-González, M. L., Castillo-Reyes, F., Flores-Gallegos, A. C., & Rodríguez-Herrera, R. (2019). Definitions and regulatory perspectives of dietary fibers. En Dietary Fiber: Properties, Recovery and Applications (pp. 1-25). Academic Press.
Den Besten, G., van Eunen, K., Groen, A. K., Venema, K., Reijngoud, D. J., & Bakker, B. M. (2013). The role of short-chain fatty acids in the interplay between diet, gut microbiota, and host energy metabolism. Journal of Lipid Research, 54 (9), 2325-2340.
Dreher, M. L. (2018). Dietary Fiber in Health and Disease. Humana Press.
Fissore, E. N., Santo Domingo, C., Gerschenson, L. N., & Giannuzzi, L. (2015). Study of the effect of dietary fiber fractions obtained from artichoke (Cynara cardunculus L. var. scolymus) on the growth of intestinal bacteria associated with health. Food & Function, 6 (5), 1667-1674.
Food and Agriculture Organization of the United Nations (FAOSTAT). (2021). FAOSTAT statistical database CROPS. http://www.fao.org/faostat/en/#data/QC/visualize
Hung, T. V., & Suzuki, T. (2018). Short-chain fatty acids suppress inflammatory reactions in Caco-2 cells and mouse colons. Journal of Agricultural and Food Chemistry, 66 (1), 108-117.
Kim, L., Kim, Y., Kwon, O., & Kim, J. Y. (2017). Antioxidant activities of ethanolic and acidic ethanolic extracts of astringent persimmon in H2O2-stimulated Caco-2 human colonic epithelial cells. Food Science and Biotechnology, 26 (4), 1085-1091.
Lambert, J. E., Parnell, J. A., Tunnicliffe, J. M., Han, J., Sturzenegger, T., & Reimer, R. A. (2017). Consuming yellow pea fiber reduces voluntary energy intake and body fat in overweight/obese adults in a 12-week randomized controlled trial. Clinical Nutrition, 36 (1), 126-133.
Lépine, A., & De Vos, P. (2018). Synbiotic Effects of the Dietary Fiber Long-Chain Inulin and Probiotic Lactobacillus acidophilus W37 Can be Caused by Direct, Synergistic Stimulation of Immune Toll-Like Receptors and Dendritic Cells. Molecular Nutrition & Food Research, 62 (15), 1800251.
López-Marcos, M. C., Bailina, C., Viuda-Martos, M., Pérez-Álvarez, J. A., & Fernández-López, J. (2015). Properties of dietary fibers from agroindustrial coproducts as source for fiber-enriched foods. Food Bioprocess Technology, 8, 2400-2408.
López-Vargas, J. H., Fernández-López, J., Pérez-Álvarez, J. A., Viuda-Martos, M. (2013). Chemical, physico-chemical, technological, antibacterial and antioxidant properties of dietary fiber powder obtained from yellow passion fruit (Passiflora edulis var. flavicapa) co-products. Food Research International, 51, 756-763.
Lucas-González, R., Viuda-Martos, M., Pérez-Álvarez, J. A., & Fernández-López, J. (2018). Evaluation of particle size influence on proximate composition, physicochemical, techno-functional and physio-functional properties of fluors obtained from persimmon (Diospyros kaki Thunb.) coproducts. Journal of the Science of Food and Agriculture, 98 (2), 504-510.
Martínez-Las Heras, R., Landines, E. F., Heredia, A., Castelló, M. L., & Andrés, A. (2017). Influence of drying process and particle size of persimmon fibre on its physicochemical, antioxidante, hydration and emulsifying properties. Journal of Food Science & Technology, 54 (9), 2902-2912.
Rodríguez-Garayar, M., Martin-Cabrejas, M. A., & Esteban, R. M. (2017). High hydrostatic pressure in astringent and non-astringent persimmons to obtain fiber-enriched ingredients with improved functionality. Food Bioprocess Technology, 10 (5), 854-865.
Song, M., Yang, G., Hoa, T. Q., Hieu, H. D., Syafiza, A., & Mohd Amin, A. S. (2019). Anti-obesity effect of fermented persimmon extracts via activation of AMP-activated
Sungsoo Cho, S., & Dreher, M. L. (Eds). (2001). Handbook of Dietary Fiber. Food Science and Technology, 113. Marcel Dekker, Inc.: New York.
Ueda, K., Kawabata, R., Irie, T., Nakai, Y., Tohya, Y., & Sakaguchi, T. (2013). Inactivation of pathogenic viruses by plant-derived tannins: strong effects of extracts from persimmon (Diospyros kaki) on a broad range of viruses. PloS One, 8 (1): e55343.
Xiao, H. W., Pan, Z., Deng, L. Z., El-Mashad, H. M., Mujumdar, A. S., Gao, Z. J., & Zhang, Q. (2017). Recent developments and trends in thermal blanching—A comprehensive review. Information Processing in Agriculture, 4, 101-127.
Yamashita, S., Sugita-Konishi, Y., & Shimizu, M. (2001). In vitro bacteriostatic effects of dietary polysaccharides. Food Science and Technology Research, 7 (3), 262-264.
Zhu, F. (2018), Interactions between cell wall polysaccharides and polyphenols. Critical Reviews in Food Science and Nutrition, 58 (11), 1818-1831.
Kazemi, A., Soltani, S., Ghorabi, S., Keshtkar, A., Daneshzad, E., Nasri, F., & Mazloomi, S. M. (2020). Effect of probiotic and synbiotic supplementation on inflammatory markers in health and disease status: A systematic review and meta-analysis of clinical trials. Clinical Nutrition, 39 (3), 789-819.

### DETAILED DESCRIPTION OF THE INVENTION

The persimmon by-product was subjected to a solvent-assisted purification or extraction process. After removal of these solvents and a drying process, the resulting fiber was exposed to homogeneous populations of beneficial microorganisms, which may be present in the human microbiome, as well as in probiotic form in foods or supplements.

The polysaccharides of the present invention are not particularly limited and, despite being subjected to a purification or assisted extraction process, presented a high concentration of covalently bound bioactive compounds, and, for example, carotenoids and phenols were found.

Dietary Fiber from Persimmon by-Product was Purified as Follows:

The persimmon by-product used for the production and purification of dietary fiber came from the residue of persimmon industrialization to produce cloudy juice.

From the industrialization of persimmon, the by-products obtained are the skin, peduncle, pulp and seeds, distributed in two groups of by-products, 1 and 2.

The Biological Studies were:

In vitro digestion: stability of bioactive compounds (total phenols and carotenoids) and their antioxidant activity. Evaluation of their fermentability: proliferation of probiotics by plate count; selectivity with control enteric microorganism by plate count; evaluation of prebiotic activity against another carbon source (glucose). Evaluation of the effect of polysaccharides on pathogens: bacteriostatic activity determined by microdilution and agar diffusion using laminar flow cabinet and incubators; determination of percentage inhibition with tetrazolium chloride (TTC) and plate count. Determination of cytotoxicity by colorimetry with crystal violet (CVS) and 3-(4,5-dimethylthiazolyl-2)-2,5-diphenyltetrazolium bromide (MTT).

In the present invention, all amounts, proportions, or percentages will always be referred to sample weight, unless otherwise specified.

The form of administration of polysaccharides in the digestion process of the present invention, may contain substances other than purified persimmon polysaccharides. For example, there may be higher proportion of phenols or carotenoids according to the purification procedure carried out from 0.005 to 80%. In this sense, it is desirable that these substances are at a concentration ranging from 0.01 to 90%.

When polysaccharides are subjected to a digestion process, they are expected to withstand the digestion process, which involves the action of enzymes and pH changes, which can alter a food. According to the present invention, the digestion process was determinant in significantly increasing the effects manifested by these polysaccharides, as compared to the same undigested.

The period in which the effects of the present invention can be obtained after ingestion is not particularly limited, but it is desirable that the number of days ingested per year be daily or close to 365 days.

The objects to which the present invention can be administered are not particularly limited, but preferably, the evaluation was performed on homogeneous communities of microorganisms.

The set of purified persimmon industrial by-product polysaccharides of the present invention can be employed as an ingredient in the preparation of different types of foods, beverages and supplements for specific uses such as in the field of health, functional foods, and parapharmacy food products.

In the present invention a functional food is understood as a food which is consumed as part of a normal diet and which contains biologically active ingredients, which offer health benefits and reduce the risk of suffering chronic diseases.

In the present invention, food supplements are understood as those foods whose purpose is to supplement the normal diet and which consist of a concentrated source of nutrients (vitamins and minerals) or of other substances having a nutritional or physiological effect, in simple or combined form.

In the present invention, parapharmacy food products are understood as those food products which, not being medicines, are consumed and made available to the users, in conformity and in accordance with what is established in the specific technical-sanitary regulations of the different categories of products existing in the market, as well as in the general regulations in force on the matter.

Throughout the description and claims, the word “comprises” and its variants are not intended to include other technical features, components or steps. To those skilled in the art, other objects, advantages, and features of the invention will be apparent in part from the description and in part from the practice of the invention.

### EXAMPLES OF REALIZATION

Being a fruit with a high concentration of sugars, which can influence the process of obtaining the dietary fiber extract. Therefore, an initial step was to wash the raw material to obtain a product with a low amount of sugars.

The water in the raw material is an important factor for the subsequent extraction process. Industrial extraction is generally carried out as a material because it reduces the complications that can occur during the extraction stage. Water removal can be carried out by using vacuum ovens at a temperature between 55 to 70° C. for 30 to 300 min or by fluidized bed with dry air for 30 to 300 min.

For the extraction and purification of fiber from the sample, the industrial by-product of persimmon is used in a solid-phase extraction process with organic solvents. These should allow the extraction of easily extractable compounds such as organic acids, remnant sugars, phenols, carotenoids, etc. For this, an aqueous, hydroalcoholic (96%) or hydroacetonic (Acetone 99.5%) medium is used and kept under agitation and ultrasound (15 to 60 min and pH between 1.5 to 4.5) to increase the efficiency of the process at a temperature between 25 and 70° C., with a solid-liquid ratio between 1:3 and 1:7. The suspension is filtered and the solid fraction is subjected to dehydration between 25-60° C. by tray drying or fluidized bed drying.

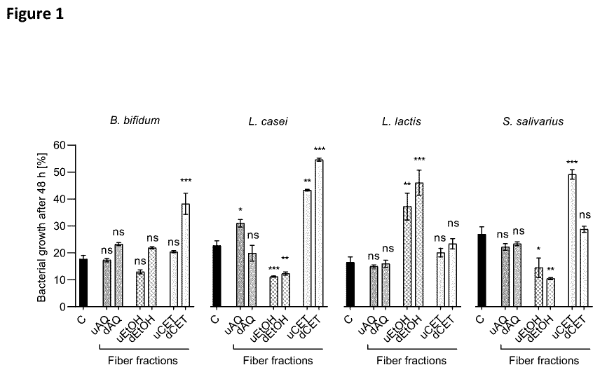

After the samples are obtained, they are exposed to microorganisms considered beneficial (Bifidobacterium bifidum, Lactobacillus casei, Lactococcus lactis subsp. lactis and Streptococcus salivarius subsp. thermophilus). These microorganisms have demonstrated a high affinity for persimmon polysaccharide fractions, with a considerable increase in their viability compared to other carbon sources such as glucose (FIG. 1).

Fiber Composition Obtained from Persimmon.

For the analysis of total, soluble and insoluble dietary fiber content, the gravimetric-enzymatic method established by AOAC 991.43 (AOAC, 2012) was used. The application of polar or apolar solvents will directly influence the properties of these fractions.

On the other hand, the extraction and quantification of compounds bound to the fiber matrix was performed by applying an alkaline hydrolysis and acidified with NaOH (2M) and HCl. The soluble fractions were analyzed for total phenol content by the Folin-Ciocalteau's method, total carotenoids, and antioxidant capacity by the ABTS radical, the results of which are shown in Table 1.

The fiber fractions were subjected to an in vitro digestion process to determine the bioaccessibility and recovery of their antioxidant compounds, as well as to determine their effect on pathogenic microorganisms.

### EXAMPLES

Each assay performed consisted of three replicates each.

In all the examples carried out on microbial cells, the procedure is the same. Fiber samples were put in interaction with homogeneous populations of bacteria by 48 to analyze their selectivity, i.e., their ability to serve as a carbon source for beneficial communities and to limit the growth of pathogens. These microorganisms were exposed to 3% w/v fiber. On the other hand, another carbon source was used as a control, which in this case was glucose, at the same concentration. The enteric Escherichia coli strain was used as a negative control to determine the selectivity of persimmon fiber. Glucose served as an equal negative control.

The interaction of persimmon fiber with other homogeneous pathogenic populations was performed for 24 hours to analyze its effect on these types of microorganisms. The amount of fiber varied from 0.09 to 50 mg/mL. The effects observed were evaluated in greater detail on resistant forms of microorganisms (biofilms, at different stages of their formation). The combination of fiber with antibiotics was explored as a potential excipient in this type of treatment, by means of the fractional index of inhibitory concentration.

To test the effects resulting from the interaction of persimmon fiber with microorganisms, as well as the stability of its components, the samples were subjected to an in vitro digestion process. Afterwards, the interaction of fiber with microorganisms was re-evaluated and its composition of compounds bound to its matrix was quantified. The quantification of the bound compounds was performed equally after fermentation with beneficial bacteria that showed affinity with the fiber samples (B. bifidum, Lb. casei, Lc. lactis subsp. lactis and S. salivarius subsp. thermophilus).

### Example 1

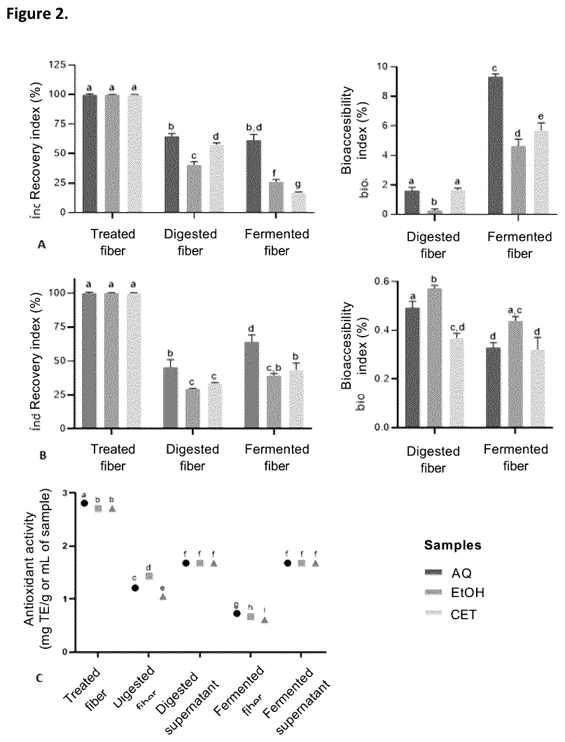

The recovery and bioaccessibility of phenolic and carotenoid biocompounds as well as their antioxidant potential are compiled in FIG. 2. Regarding the content of total phenols (FIG. 2A), a decrease in their recovery is recorded after digestion, but their recovery increases after probiotic fermentation (p<0.05). Carotenoid content (FIG. 2B) presented a significant decrease (p<0.05) of its content upon digestion, but with an increase after fermentation (p<0.05). Purification by aqueous solvent generated the highest bioaccessibility and recovery of these compounds. The antioxidant capacity of persimmon fiber presented a similar behavior to its phenol and carotenoid content (FIG. 2C), with a higher expression of this activity in the digestion process than in fermentation (p<0.05).

### Example 2

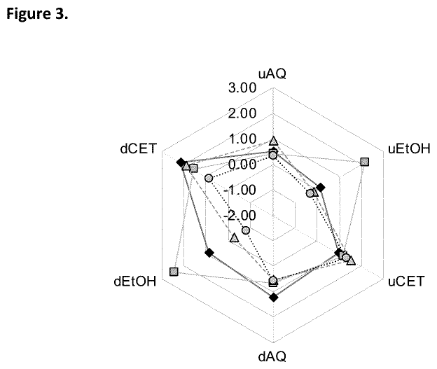

Selective effect of persimmon fiber on microorganisms. FIG. 3 reflects that, in general, fiber samples are a preferential carbon source for the beneficial microorganisms tested than other carbon sources such as glucose. The calculated prebiotic activity assessment indicates that the higher the positive value, the higher the affinity of that substrate and a particular microorganism and, in turn, limits the growth of pathogens such as E. coli; especially, after in vitro digestion performed.

### Example 3

Effect of persimmon fiber on pathogenic microorganisms. The pathogens were not inhibited by the persimmon fiber samples by more than 40%; however, their population density did not increase compared to the control (Table 2). The population of the pathogens evaluated showed a tendency to decrease their population density (p<0.05). Gram-positive bacteria (Staphylococcus aureus, Bacillus cereus and B. subtilis) were the most sensitive to these samples (p<0.05). This effect was markedly observed at concentrations of 4 log CFU/mL of Gram-positive and 8 log CFU/mL of Gram-negative bacteria.

### Example 4

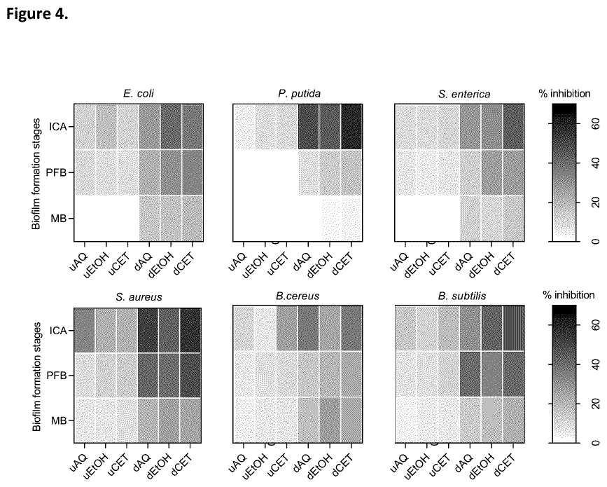

Effect of persimmon fiber on the three stages of bacterial biofilm formation. The results obtained showed that persimmon fiber, after digestion, generated a significant (p<0.05) cell anchorage prevention effect for biofilm formation (FIG. 4). The initial cell anchorage inhibition was more than 50% in Pseudomonas putida, S. aureus and B. subtilis, after being exposed to the fiber samples purified by aqueous, hydroethanolic and hydroacetonic solvents.

### Example 5

Synergistic activity of digested persimmon fiber with antibiotics (Table 3). The effect of fiber combination with broad-spectrum antibiotics with poor efficacy was evaluated with additive effects of the combination of persimmon fiber and kanamycin or persimmon fiber and gentamicin. In addition, synergistic combinations of persimmon fiber with kanamycin (FICI: 0.38±0.00) to inhibit B. subtilis and persimmon fiber with gentamicin (FICI: 0.38±0.04) against S. aureus were identified. These combinations were tested in a 24 h kill time assay with sub-inhibitory concentrations of the compounds alone and in combination (FICI). The combination of fiber with gentamicin generated a bactericidal effect on S. aureus. The rest of the combinations generated a bacteriostatic effect (Table 4).

1. Dietary fiber derived from persimmon by-products characterized in that it is obtained by means of a purification or extraction process assisted with acetone as solvent.

2. Dietary fiber according to claim 1 characterized in that it is obtained by means of a purification or extraction process assisted with a solution of acetone in water at 70% by volume of acetone as solvent.

3. Dietary fiber according to any preceding claim characterized in that it is obtained by a purification or assisted extraction process with 5:1 ratio by weight of persimmon by-products to solvent.

4. Dietary fiber according to claim 3 characterized in that it is obtained by a purification or assisted extraction process in which the persimmon by-products with the solvent are subjected to heating at 60° C. for 15 minutes in a vessel with stirring of at least 3000 rpm and said stirred digestion step is repeated three times.

5. Dietary fiber according to claim 4 characterized in that the mixture resulting from the purification or assisted extraction step is filtered, the solvent is removed from the solid obtained, and the resulting material is frozen at −20° C.

6. Dietary fiber according to claim 5 characterized in that the frozen material is freeze-dried, ground, and sieved with a 0.5 mm sieve.

7. Process for obtaining dietary fiber characterized in that it comprises a step purification or assisted extraction of persimmon by-products with an acetone solution as solvent.

8. Process according to claim 7 characterized in that the purification or extraction step is carried out with a solution of acetone in water at 70% by volume of acetone as solvent.

9. Process according to claim 8 characterized in that the purification or assisted extraction step is carried out with 5:1 ratio by weight of persimmon by-products and solvent.

10. Process according to claim 9 characterized in that the purification or assisted extraction step comprises subjecting the persimmon by-products with the solvent to a purification or assisted extraction process at 60° C. for 15 minutes in a vessel with agitation of at least 3000 rpm and said step is repeated three times.

11. Process according to claim 10 characterized in that the mixture resulting from the purification or assisted extraction step is filtered, the solvent is removed from the solid obtained, and the resulting material is frozen at −20° C.

12. Process according to claim 11 characterized in that the frozen material is freeze-dried, ground and sieved with a 0.5 mm sieve.

13. Dietary fiber as obtained according to claim 12 is mixed with microorganisms considered beneficial (Bifidobacterium bifidum, Lactobacillus casei, Lactococcus lactis subsp. lactis and Streptococcus salivarius subsp. thermophilus), at a concentration of 10⁷ CFU/mL, manage to control and prevent the proliferation of Escherichia coli after 48 hours of incubation at 37° C. in aerobiosis and/or anaerobiosis.
